(19) United States
(12) Patent Application Publication
(10) Pub. No.: US 2021/0259704 A1
(43) Pub. Date: Aug. 26, 2021

(54) METHODS AND SYSTEMS FOR CONTROLLING TOURNIQUETS

(71) Applicants: James S. Stewart, (US); Christopher Green, (US); Ashish Shah, (US); Marturion Electronics Limited, Lisburn (GB)

(72) Inventors: Andrew Barr, Annahilt (GB); James S. Stewart, Baileyton, AL (US); Christopher Green, Moody, AL (US); Ashish Shah, Vestavia, AL (US)

(21) Appl. No.: 17/032,130

(22) Filed: Sep. 25, 2020

(30) Foreign Application Priority Data
Feb. 26, 2020 (GB) 2002724.9

(51) Int. Cl. A61B 17/135

(52) U.S. Cl. CPC A61B 17/1355; A61B 2017/00022

(57) ABSTRACT

A method and system for controlling a tourniquet establishes a set of values for a characteristic of an induced pressure pulse in the tourniquet while the tourniquet is inflated to a working pressure which is at a predetermined offset above the limb occlusion pressure. Maximum and minimum thresholds for this characteristic value are established, such that during the working phase of application of the tourniquet the induced pressure pulse is continually monitored and the value of the characteristic is compared to the thresholds. If a threshold is reached the tourniquet is inflated or deflated accordingly to restore the value of the characteristic to within the thresholds. The characteristic of the induced pressure pulse, which may be e.g. pulse height, area under pulse or pulse width at a percentage of peak height, may also be used to determine behaviour of the pressure pulse by noting a variation in the rate of change as the tourniquet is inflated past the limb occlusion pressure.

### FIELD OF THE INVENTION

The present invention relates to methods and systems for controlling tourniquets and similar devices for restricting blood flow in a body part, and in particular to such devices operated by inflation using a pressurised fluid.

### BACKGROUND OF THE INVENTION

Tourniquets used in surgery are designed to occlude blood flow to a body part (most usually a limb) during the surgical procedure. They are also used in emergency trauma situations to reduce blood loss from a traumatic injury.

Tourniquets can be manually inflated, or can be inflated under the control of an automated controller. Tourniquets with automatic pressure control of this type are referred to herein as “smart tourniquets”.

The specification for a smart tourniquet is relatively simple—it should provide an occlusion to blood flow into a limb (that is an arm or a leg) during surgery in order to prevent bleeding (or if used for treating trauma in an emergency, it should provide an occlusion to prevent excessive blood loss). The means of occlusion is typically a pneumatically inflatable cuff fitted around the proximal end of the limb. The designation “smart” means that the pressure to which the cuff is inflated should be determined by some measurement of patient vital signs and thereby tailored to the patient rather than simply one-pressure-fits-all.

In contrast, non-smart tourniquets are merely inflated to an excess of pressure to which the patient systolic pressure is unlikely ever to reach and left at this pressure for the duration of the surgery. This approach ensures that there is no bleeding during the surgery but can lead to many post-operative complications, the least of which is bruising.

Systolic pressure is also known as “limb occlusive pressure (LOP)” in the context of the tourniquet and is defined as the pressure needed in the tourniquet in order to adequately occlude blood flow. It is well known however that LOP changes in the course of surgery; most commonly it reduces as a result of anaesthesia, so that even if tourniquet pressure is set at a reasonable pressure at the start of surgery when LOP is normal, this is no guarantee that it will not be excessive later in the surgical procedure when LOP may have dropped substantially.

Conventional smart tourniquets operate to maintain the tourniquet pressure at a set value, relative to the LOP occlusion pressure which is determined in advance of surgery in conventional fashion. This means that if the LOP drops or increases then the pressure may be excessive leading to tissue damage and post-operative pain, or alternatively it may be too low resulting in the tourniquet being ineffective to occlude flow to the surgical site.

### SUMMARY OF THE INVENTION

In accordance with one aspect of the invention there is provided a method of controlling a tourniquet comprising the steps of:
(a) performing an inflation phase in which a tourniquet applied to a body part is inflated to a working pressure which exceeds the limb occlusion pressure (LOP) of said body part by an offset amount;
(b) measuring the time-varying pressure induced in said tourniquet by the vascular system of said body part during said inflation;
(c) deriving from said time-varying pressure a set of values for a characteristic of the induced pressure pulse in said tourniquet at a plurality of tourniquet pressures during the inflation phase;
(d) determining from said set of values a first threshold value intermediate between the value of the characteristic at the LOP and the value of the characteristic at the working pressure;
(e) monitoring the value of said characteristic during a working phase of the tourniquet while maintaining said working pressure; and
(f) upon determining that the value of said characteristic has reached or passed said first threshold, increasing the working pressure of the tourniquet.

A change in LOP will result in a corresponding change in the induced pressure pulse in the tourniquet. By observing the patient's induced pressure pulse in the tourniquet during inflation, in particular as the tourniquet is inflated past the LOP, the behaviour of the induced pressure pulse can be accurately predicted in the event of a change in LOP (e.g. the offset between the tourniquet pressure and the LOP decreasing or increasing).

Therefore, by setting a threshold for a value of a characteristic of the induced pressure pulse—as a purely non-limiting example, this characteristic could be the area under the pulse after normalisation of all pulses to a common height—the threshold can be correctly adapted to provide a prediction for when further inflation of the tourniquet is required to prevent bleeding in the event of LOP rising for that patient. The threshold is based on a set of values of the characteristic of the induced pressure pulse observed during inflation, and is thus personalised to the patient and a reliable indication of the need to inflate.

The “first threshold” may be a value that is less than the value of the characteristic at the working pressure (e.g. if the characteristic exhibits a rise between the LOP and the working pressure), in which case it is referred to herein as a “min threshold”. Alternatively, it may be greater than the value of the characteristic at the working pressure, if the characteristic falls between the LOP and the working pressure, in which case it is referred to herein as a “max threshold”.

It is possible either for the thresholds to be determined individually for that patient or for the observed variation in the characteristic to be matched against a model and for thresholds associated with the matching model to be applied.

Preferably, the increase of pressure in step (f) is sufficient to restore the value of the characteristic so that it no longer breaches the first threshold.

Preferably, step d) further comprises:
determining from said set of values a second threshold value wherein the value of the characteristic at the working pressure is intermediate between the second threshold value and the value of the characteristic at the LOP; and

and the method further comprises:
(g) upon determining that the value of said characteristic has reached or passed said second threshold, decreasing the working pressure of the tourniquet.

The “second threshold” may be either a min threshold or a max threshold, depending again on the behaviour of the selected characteristic. It will be appreciated that if the first threshold is a min threshold (because the value of the characteristic rises from LOP to the working pressure), the second threshold will in general be a max threshold and vice versa.

It is however envisaged that both thresholds could be “max thresholds” or “min thresholds” if the graph of the characteristic is at a local minimum or local maximum when at the working pressure, provided that the shape of the graph enables a reliable differentiation between the tourniquet pressure becoming too close to the LOP or too far from the LOP. This could for example be the case where the characteristic rises steeply or abruptly towards the second threshold but gradually towards the first threshold, or it some other change in behaviour of the characteristic is observed when the LOP rises or falls towards or away from the working pressure. It is also possible that the system can differentiate by observing a second characteristic of the pressure pulse that is indicative or confirmatory of the direction in which the LOP is moving relative to the tourniquet working pressure.

Preferably, the decrease of pressure in step (g) is sufficient to restore the value of the characteristic so that it no longer breaches the first threshold.

Suitably, the increase or decrease of pressure may be sufficient to restore the value of the characteristic to a value observed at the original working pressure following the inflation phase.

The characteristic may be the area under the pulse.

The characteristic may be the area under the pulse following normalisation of each pulse to a common height.

The characteristic may be the rate of decay of pulse waveform following shoulder inflexion.

The characteristic may be the relative height of shoulder inflexion.

The characteristic may be the pulse width at a predetermined percentage of peak pulse height.

Preferably in such cases, said percentage is between 20% and 90%.

It will be appreciated that more than one characteristic may be employed in combination to increase accuracy or provide confirmation of the action required, or a characteristic may be derived from more than one metric of the induced pressure pulse.

Preferably in step (c) the plurality of tourniquet pressures at which said set of values are derived include tourniquet pressures both below and above the LOP.

By including a number of values both below and above the LOP (and preferably also at the LOP) the behaviour of the tourniquet can be more accurately modelled or fitted to an existing model, and therefore can be better adapted to the patient's physiology and cardiovascular condition, the physical characteristics of the tourniquet and the site of application.

Preferably, the method further comprises the step of determining a transition in the rate of change of said characteristic in the region of the LOP.

It has been found that the induced pressure pulse will exhibit characteristic changes in the immediate region of the LOP, observable as transitions in the graph of particular characteristics of the pulse, and these can be used to better characterise and personalise the operation of the tourniquet, particularly to prevent the tourniquet pressure being too close to the LOP.

Preferably, this determination comprises identifying a transition which is one of:
an inflection point;
a transition from one mode to another mode, wherein the modes are increasing, decreasing and constant;
a step transition between greater and lesser rates of increase or decrease, or between lesser and greater rates of increase or decrease.

Preferably, the method further comprises:
determining the LOP from the measurements of induced pressure pulse during the inflation phase.

Such a determination of the LOP can be achieved from observing a transition as noted above, or by using conventional oscillometric techniques during inflation. For example, this may involve determining the tourniquet pressure at which occurs the peak of the envelope of pulse heights, defining this as MAP (mean arterial pressure), and then subsequently determining the pressures at which this envelop of pulse heights falls below a specific fraction of the peak of the envelope. The corresponding pressure above MAP is normally termed systolic pressure but in this application is equivalent to LOP.

Preferably, following a determination of said LOP, the tourniquet is inflated as part of the inflation phase to a working pressure equal to the LOP plus a stored offset.

There is also provided a system for controlling a tourniquet comprising:
a processor having at least one output adapted to provide control signals to inflate and deflate a tourniquet, and at least one input to receive data indicating pressure in the tourniquet from a sensor the processor being programmed with instructions which when executed are effective to perform the method steps disclosed herein.

Such a system can be implemented using a processor that is a dedicated, programmed processor designed to carry out the method, forming part of an instrument having a working memory, permanent storage, and inputs and outputs to the memory and storage, and optionally user inputs and displays.

The system can also be implemented using a processor which is part of a general purpose computer with suitable programming, or can be implemented using a circuit designed with logic to implement the method steps.

There is also provided a computer program product comprising stored instructions which when executed in a processor having outputs adapted to provide control signals to inflate and deflate a tourniquet, cause said processor to perform the method steps disclosed herein.

### BRIEF DESCRIPTION OF THE DRAWINGS

The invention will now be further illustrated by the following description of embodiments thereof, given by way of example only with reference to the accompanying drawings, in which:

FIG. 1 is a block diagram of a system for controlling a tourniquet;

FIG. 2 is a flowchart of a method of controlling a tourniquet during an inflation phase;

FIG. 3 is a flowchart of a method of controlling a tourniquet during a working phase;

FIG. 4 is a graph of induced pressure signal in a tourniquet over time as the tourniquet pressure was decreased from a higher value to a pressure substantially below the LOP;

FIG. 5 is a graph of the data of FIG. 4 with the height of each pressure peak normalised;

FIG. 6 is a graph of the pulse width at 80% of the peak pulse height against time for the data of FIG. 5;

FIG. 7 is a graph of the area under the pulse against time for the normalised pulses of FIG. 5; and

FIG. 8 is a graph of the area under the pulse against time for the non-normalised pulses of FIG. 4.

### DETAILED DESCRIPTION OF PREFERRED EMBODIMENTS

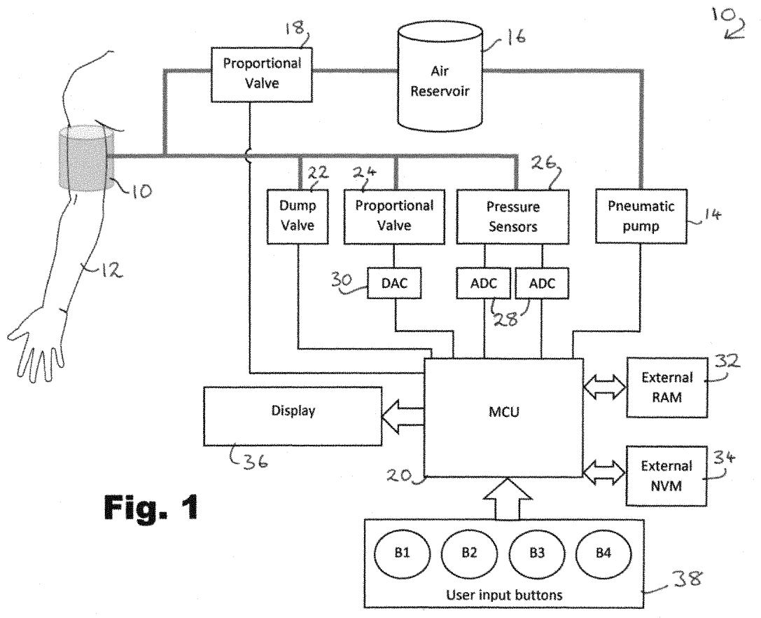

In FIG. 1 there is shown a block diagram of a system for controlling a tourniquet 10 applied to a limb 12. In this diagram air lines are shown using heavier weights and electrical connections shown using regular weight lines, while wide arrows indicate input/output (I/O) buses.

The tourniquet 10 is of the inflatable cuff type and can be pressurised by means of a pneumatic pump 14 which feeds an air reservoir 16 to store air under pressure, with a proportional valve 18 being controllable by a microcontroller unit (MCU) 20.

Air can be released using a dump valve 22 or a proportional valve 24. The proportional valve 24 is primarily used as the controlled deflation mechanism for the tourniquet, while the dump valve 22 is provided for rapid deflation such as in an emergency.

The pressure of air in the tourniquet 10 is monitored by a plurality of pressure sensors 26 (provided for redundancy in case of failure), whose output is digitised by analog-to digital converters (ADCs) 28 and passed to the MCU 20. The MCU is programmed to perform signal analysis on the time-varying pressure signal(s) from the sensor(s) to derive key characteristics of the induced pressure waveform such as the signal height, pulse width, area under the pulse, and so on. It will be appreciated that the same functionality can be implemented by a dedicated integrated circuit, a programmable microcontroller, or a general-purpose computer having an interface to receive the ADC output.

The MCU provides output signals to control the operation of the pneumatic pump 14, the proportional valves 18, 24 and the dump valve 22 (via a digital-to-analog converter, DAC, 30), and thereby to regulate the pressure instantaneously in the tourniquet as described further below.

The MCU is also in communication with a working memory or RAM 32 and a non-volatile memory 34, as well as a display 36 and a number of user input buttons 38 shown as buttons B1, B2, B3, B4, which can be used by a user of the system to control the operation of the system. It will be appreciated that four buttons are shown for illustrative purposes only, and any number of suitable buttons can be provided according to the intentions of the system designer. The buttons may be physical buttons with dedicated functions (e.g. STOP, START, CANCEL, CONFIRM, UP, DOWN), or may be buttons whose functions are dependent on the current state of operation, e.g. with the function being displayed above the buttons on the display screen. It is also possible for a different type of interface to be provided, such as a touchscreen displaying buttons or other user controls with which a user may interact, a keypad, or dials and sliders etc.

Referring now to FIG. 2, a flowchart of the initial operation of the system during an inflation phase is illustrated. After the system starts, step 50, the MCU performs a system initialisation, step 52, during which it may read program instructions and stored variables from NVM, and initialise the various connected components shown in FIG. 1 in a conventional manner, such as by closing all valves and causing the pump 14 to pressurise the air reservoir 16 to a desired pressure.

The system initialisation also triggers a subroutine 54 which runs constantly in the background as long as the system is operational. This subroutine monitors for a CANCEL button (which may be one of the buttons B1-B4) being pressed, and if CANCEL is pressed, step 56, a rapid deflation of the tourniquet is performed immediately, step 58. This provides an important safety feature to prevent injury if the tourniquet is overpressurised, or pressurised prematurely, etc.

After system initialisation the user is prompted via the display to enter an offset value, which is the desired degree of overpressure to be maintained above the limb occlusion pressure or LOP, step 60. The system pauses, step 62, until an offset has been entered. The user may enter the offset using any suitable and provided interface, such as by using up and down buttons to adjust a numeric value displayed on the display, followed by an enter button to confirm.

Once an offset is entered, the system optionally validates that the offset is a valid value, and then waits for a START input, step 64. When START is pressed, step 66, the system initiates cuff inflation, step 68, by sending a signal to open proportional valve 18, and allow air to enter the tourniquet 10 from the reservoir 16.

The MCU then starts to monitor the induced pressure pulse in the tourniquet, step 70 and to store the pulse data. As the pressure sensor outputs are digitised, the pulses are recorded as a time series of data points representing the instantaneous output from the pressure sensor.

Characteristic values of the induced pressure pulse are calculated from the instantaneous received pressure values from the sensors, and an analysis is performed to determine if those values indicate that the tourniquet pressure has reached the LOP, either using standard oscillometry techniques or by identifying a transition in the rate of change of a measured characteristic of the induced pressure pulse.

When the LOP is reached, step 72, the LOP value is stored, and the tourniquet is then inflated up to a tourniquet pressure (TP) equal to the LOP plus the offset entered by the user, step 74. It is possible, in other implementations, to skip the user entering the offset and use a default value, but for surgical applications the user is likely to want to be able to control the offset according to clinical and surgical needs.

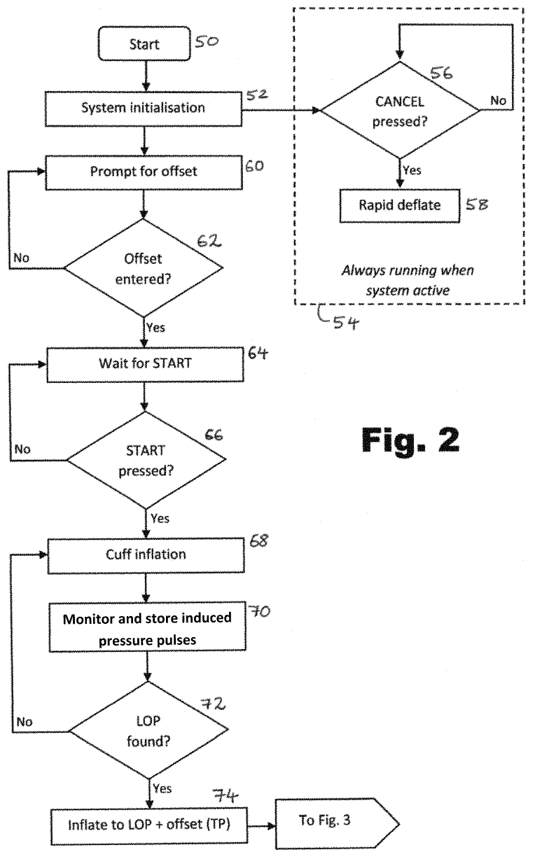

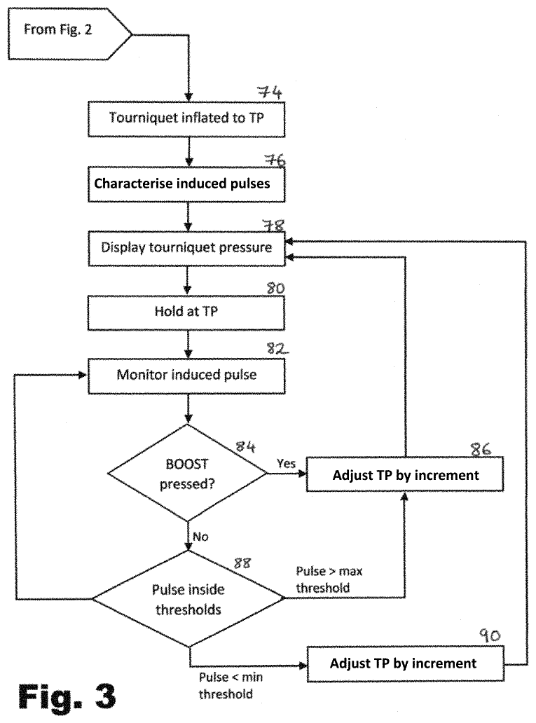

Referring to FIG. 3, the working phase of operation of the tourniquet is shown in flowchart form. The flowchart starts at step 74, which has the tourniquet inflated to the desired pressure TP. The MCU then characterises the stored pulse data recorded up to this pressure, step 76, by measuring one or more appropriate characteristics of the induced pressure pulses during the inflation up to and beyond LOP. This characterisation provides a reference of pulse shape as a function of pressure relative to LOP, describing the behaviour of that particular patient's pulse shape over a range of pressure above and below LOP. This characterisation also provides a value that is indicative of the functioning of the tourniquet at the working pressure for this specific patient. It will be appreciated that different patients will exhibit different pulse characteristics, according to their physiology, the site of application of the tourniquet, their clinical condition and various other factors. By characterising the patient's induced pressure pulse in the tourniquet at a known “good” tourniquet pressure (namely the LOP plus a desired offset or overpressure), the tourniquet's inflation can be maintained and controlled.

In step 78 the current tourniquet pressure is displayed. The MCU controls the pump and the proportional valves to maintain the tourniquet at this desired pressure TP, step 80, until a change in pressure is determined to be required. During the operation, the MCU continually monitors the induced pressure in the tourniquet, step 82, by performing a signal analysis to derive the instantaneous value of whatever characteristic was selected to characterise the tourniquet's response at the LOP plus offset pressure.

If the surgeon (or other user) wishes to increase the offset or overpressure above LOP at any time, this can be done using a BOOST button, step 84. If the button is pressed the MCU increases the stored offset and increases the tourniquet pressure TP accordingly. The amount of boost pressure applied may be a predetermined increment or may be a value input by the user.

Assuming the BOOST button is not pressed, the MCU determines if the pressure pulse characteristic lies between threshold values. These values are based on the value used to characterise the pulse in step 76, and are specified as a min threshold and a max threshold. The min threshold is a value for the characteristic that is below the current value exhibited at the tourniquet working pressure, and the max threshold is a higher value.

Depending on what characteristic value is chosen, and whether this value has been observed to decrease or increase as the tourniquet pressure approaches LOP, the min threshold may be indicative of a need to either increase or decrease pressure, and similarly the max threshold may be indicative of a need to increase or decrease tourniquet pressure.

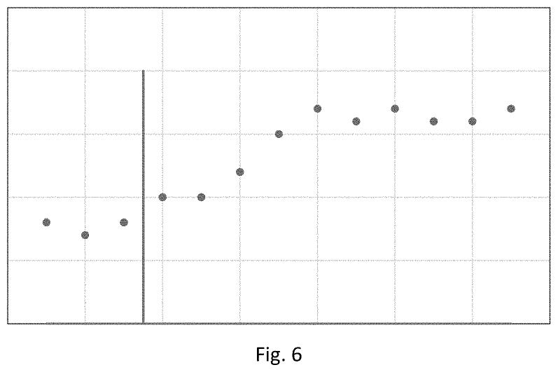

For example, if the characteristic value being used to monitor the operation of the tourniquet is the pulse width at 80% of pulse peak height (as shown and discussed below with respect to FIG. 6), this value is observed to decrease as the pressure in the tourniquet increases from LOP to TP. Assuming that the 80% pulse width was observed to be 120 (arbitrary units) at the LOP and 100 at the TP (=LOP+offset pressure), then the min and max threshold values might be set at e.g. 90 and 110. During normal operation the pulse width will vary naturally from pulse to pulse but as long as the LOP remains constant the variation should like between these thresholds.

However if the patient's LOP increases, the induced pressure pulse in the tourniquet will change shape and the observed 80% pulse width value will increase as a result of this change, moving from 100 towards 120 (the value which it would be expected to reach at the point where the tourniquet pressure was just equal to LOP leading to a risk of bleeding past the tourniquet). Therefore if the 80% pulse width breaches the max threshold of 110, step 84, remedial action is initiated by adjusting the tourniquet pressure by an increment, step 86, specifically by increasing the tourniquet pressure in this example, and thereby restoring some or all of the offset and leading to a reduction in the pulse width as a result.

The manner of increasing the pressure, and the amount of the increase, is at the choice of the system designers. For example, the system may be configured to simply apply a preset amount of pressure increase, or it can perform a gradual increase e.g. until the pulse width is observed to have been restored to its initial value or some other value deemed acceptable.

Conversely if the patient's LOP drops, then the tourniquet pressure might be inappropriately high leading to possible tissue damage, injury or post-operative complications, and this would be observed as a decrease in the 80% pulse width. If the 80% pulse width decreases to the point that it breaches the min threshold of 90, step 88, then the system responds by adjusting the pressure in the tourniquet, step 90, to decrease the tourniquet pressure by an increment (or under feedback control until an appropriate value is observed).

While the above description uses the specific example of observations of the 80% pulse width, which displays a reducing value as the offset from LOP increases, other characteristic metrics derived from the pulse may show an increase as the offset from LOP increases, and in such cases, a breach of the min threshold would lead to an upward adjustment of tourniquet pressure and vice versa for the max threshold.

Logic in the MCU programming may require stronger evidence for decreasing the tourniquet pressure than it does for increasing the tourniquet pressure: it is more important to react promptly to any risk of bleeding promptly and at the first indication that the tourniquet may be inadequately pressurised, and for similar reasons one may be cautious about reducing tourniquet pressure lest this induces bleeding.

The system continues in a loop, displaying the tourniquet pressure, monitoring the induced pulse, and monitoring the BOOST button as well as the induced pressure pulse for an indication that the tourniquet pressure TP needs to be increased or decreased, until the user stops the process (not shown) e.g. by pressing a STOP button which causes the tourniquet to deflate in a controlled manner.

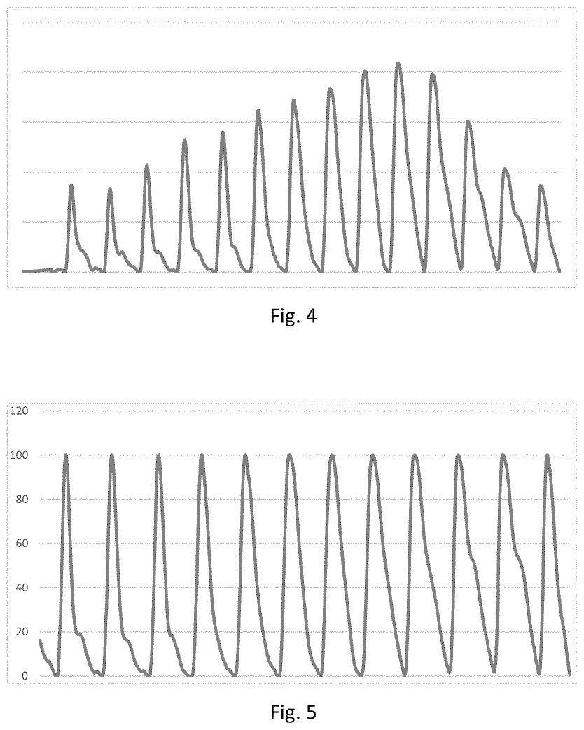

FIG. 4 is a waveform of the induced pressure pulse measured in an experimental setup using a Fluke ProSim 8 vital signs simulator to mimic a human subject. A tourniquet cuff was applied to a dummy arm, and the simulator used to induce a pressure pulse in the tourniquet, mimicking the induced pressure pulse observable in a human subject when blood flow to a limb is occluded with a tourniquet. The systolic pressure was adjusted up and down to mimic limb-occlusive pressure rising and falling.

FIG. 4 shows the induced pressure signal (Y-axis) over time (X-axis) as the tourniquet pressure was decreased from a higher value, shown on the graph at the left where the signal is almost zero, to a pressure substantially below the LOP at the right.

FIG. 5 shows the same data but normalised so that all pulse heights are identical—this graph emphasises more eloquently the changes in other variables that describe the shape of the pressure pulse.

From this waveform the MCU may derive numerous different characteristics of the pulse waveform including but not limited to:
pulse height
pulse area (otherwise known as the cumulative sum of the sample values that make up the pulse waveform)
pulse area from normalised pulses
rate of decay of pulse waveform following shoulder inflexion
relative height of shoulder inflexion
pulse width at 80% of peak height (or 40%, or any other desired point on the waveform height)

One can also observe typical characteristic features of the arterial pulse from the induced waveform which the skilled person will be familiar with such as the systolic upstroke, systolic peak, systolic decline, dicrotic notch, diastolic runoff, end diastolic pressure and mean arterial pressure.

It has been observed that these characteristics and pulse features display changes as the tourniquet passes the LOP, and from an analysis of the induced pressure pulse the LOP can be identified.

FIG. 6 is derived from FIG. 5 and plots the pulse widths at 80% of pulse height plotted as a function of tourniquet pressure with high pressure to the left and decreasing pressure to the right. The vertical line represents the point for which tourniquet pressure is independently determined to be equal to systolic pressure or LOP.

This graph illustrates how that for tourniquet pressure higher than LOP the pulse width is relatively constant but as tourniquet pressure falls below LOP the pulse width increases steadily.

This is only one example of the way pressure pulse waveform may be used to control tourniquet pressure.

In this specific example, and as described above with reference to FIG. 3, if the 80% pulse width is found to be greater than a threshold value then tourniquet pressure can be increased until the 80% pulse width is brought back to below this threshold. Similarly, if the 80% pulse width is found to be less than a threshold value then tourniquet pressure can be reduced until the 80% pulse width is brought back up to this threshold.

Accordingly, in the inflation phase of FIG. 2, the LOP may be identified from the 80% pulse width (for example) changing during the increase in tourniquet pressure from a decreasing mode to a levelling off (moving right to left on the FIG. 6 graph). Then the tourniquet pressure is increased by the offset pressure to provide the initial tourniquet pressure TP.

As described above, during the working phase, two thresholds are set and monitored for. The max and min values can be set very simply, as percentage offsets, or preferably they are determined as a result of the analysis of pulse shape changes that occur during the initial inflation period (the characterisation step 76, FIG. 3). By forming an understanding of how the pulse features change as a function of changes in pressure above and below LOP it is possible to normalise a given patient to a preset table of thresholds that apply to each tourniquet pressure. In other words, we may define for all patients an expected behaviour of their pulse shape at each tourniquet pressure and accordingly define the threshold values that should trigger a change in tourniquet pressure up or down. The system then adjusts those threshold values for a specific patient based on the behaviour of their pulse shape features during the inflation phase.

If either of these thresholds is reached, then the tourniquet pressure TP is incremented up or down appropriately, and in this way the overpressure is adjusted to compensate for increases or decreases in LOP dynamically and automatically. No additional sensors are required and the tourniquet is kept at all times at a safe overpressure that is sufficient to occlude blood flow but insufficient to damage the patient.

This example should not suggest that the 80% pulse width variable is any more or less significant than another in determination of the relationship between tourniquet pressure and LOP—indeed for each different patient there is found to be a different set of variables that may be used to best effect in identifying the need to adjust tourniquet pressure—this example is merely included as actual quantitative data to explain how to analyse the pressure pulse waveform to determine instantaneous LOP.

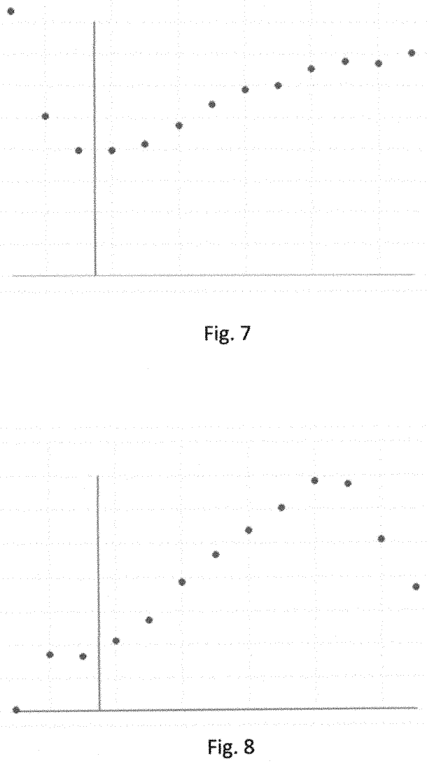

FIGS. 7 and 8 illustrate the use of another characteristic of the induced pulse, namely the area under the pulse. In FIG. 7, the area under each pulse is plotted when the pulses have been normalised to the same pulse height. In FIG. 8, the un-normalised area under each pulse is plotted. Again in each case, high pressure is to the right, and low pressure to the left.

It can be observed that at the LOP, which was independently determined, the pulse area reaches a minimum, and clearly visible in FIG. 7 there is a distinct point of inflection, which can be used to identify the LOP as the cuff is inflated. As with the 80% pulse width, the area under the pulse at the LOP+offset pressure can be characterised and thresholds derived from this area that are indicative of lower and higher desired tourniquet pressures relative to the LOP, so that if the area under the pulse reaches either threshold during the working phase the tourniquet pressure TP can be incrementally adjusted until the area again lies in a desired range indicative of the correct overpressure for the tourniquet relative to the current LOP.

Most of the above discussion has assumed complete occlusion of the artery by the tourniquet. In this case the only modulation on the tourniquet baseline pressure is that caused by the blood pressure wave itself. In the case where tourniquet pressure falls below LOP so that perfect occlusion is no longer maintained, a much more significant factor starts to affect the pressure waveform. This is because the tourniquet pressure waveform is no longer modulated solely by the arterial pressure waveform but additionally by the displacement of air in the tourniquet caused by blood flowing through the artery under the partial constraint of the tourniquet. This results in a profoundly different pressure waveform which loses much of the distinctive shape of the standard arterial pressure waveform and is an important “last resort” for detecting the risk of bleeding due to tourniquet pressure being too low to maintain perfect occlusion.

1. A method of controlling a tourniquet comprising the steps of:
(a) performing an inflation phase in which a tourniquet applied to a body part is inflated to a working pressure which exceeds the limb occlusion pressure (LOP) of said body part by an offset amount;
(b) measuring the time-varying pressure induced in said tourniquet by the vascular system of said body part during said inflation;
(c) deriving from said time-varying pressure a set of values for a characteristic of the induced pressure pulse in said tourniquet at a plurality of tourniquet pressures during the inflation phase;
(d) determining from said set of values a first threshold value intermediate between the value of the characteristic at the LOP and the value of the characteristic at the working pressure;
(e) monitoring the value of said characteristic during a working phase of the tourniquet while maintaining said working pressure; and
(f) upon determining that the value of said characteristic has reached or passed said first threshold, increasing the working pressure of the tourniquet.

2. A method according to claim 1, wherein the increase of pressure in step (f) is sufficient to restore the value of the characteristic so that it no longer breaches the first threshold.

3. A method according to claim 1, wherein step (d) further comprises:
determining from said set of values a second threshold value wherein the value of the characteristic at the working pressure is intermediate between the second threshold value and the value of the characteristic at the LOP; and
the method further comprising:
(g) upon determining that the value of said characteristic has reached or passed said second threshold, decreasing the working pressure of the tourniquet.

4. A method according to claim 3, wherein the decrease of pressure in step (g) is sufficient to restore the value of the characteristic so that it no longer breaches the first threshold.

5. A method according to claim 3, wherein the increase or decrease of pressure is sufficient to restore the value of the characteristic to a value observed at the original working pressure following the inflation phase.

6. A method according to claim 1, wherein said characteristic is the area under the pulse.

7. A method according to claim 1, wherein said characteristic is the area under the pulse following normalization of each pulse to a common height.

8. A method according to claim 1, wherein said characteristic is the rate of decay of pulse waveform following shoulder inflexion.

9. A method according to claim 1, wherein said characteristic is the relative height of shoulder inflexion.

10. A method according to claim 1, wherein said characteristic is the pulse width at a predetermined percentage of peak pulse height.

11. A method according to claim 10, wherein said percentage is between 20% and 90%.

12. A method according to claim 1, wherein in step (c) the plurality of tourniquet pressures at which said set of values are derived include tourniquet pressures both below and above the LOP.

13. A method according to claim 1, further comprising the step of determining a transition in the rate of change of said characteristic in the region of the LOP.

14. A method according to claim 13, wherein the determination comprises identifying a transition which is one of:
an inflection point;
a transition from one mode to another mode, wherein the modes are increasing, decreasing and constant;
a step transition between greater and lesser rates of increase or decrease, or between lesser and greater rates of increase or decrease.

15. A method according to claim 1, further comprising:
determining the LOP from the measurements of induced pressure pulse during the inflation phase.

16. A method according to claim 1, wherein following a determination of said LOP, the tourniquet is inflated as part of the inflation phase to a working pressure equal to the LOP plus a stored offset.

17. A system for controlling a tourniquet comprising:
a processor having at least one output adapted to provide control signals to inflate and deflate a tourniquet, and at least one input to receive data indicating pressure in the tourniquet from a sensor, the processor being programmed with instructions which when executed are effective to perform a method having the steps of:
(a) performing an inflation phase in which a tourniquet applied to a body part is inflated to a working pressure which exceeds the limb occlusion pressure (LOP) of said body part by an offset amount;
(b) measuring the time-varying pressure induced in said tourniquet by the vascular system of said body part during said inflation;
(c) deriving from said time-varying pressure a set of values for a characteristic of the induced pressure pulse in said tourniquet at a plurality of tourniquet pressures during the inflation phase;
(d) determining from said set of values a first threshold value intermediate between the value of the characteristic at the LOP and the value of the characteristic at the working pressure;
(e) monitoring the value of said characteristic during a working phase of the tourniquet while maintaining said working pressure; and
(f) upon determining that the value of said characteristic has reached or passed said first threshold, increasing the working pressure of the tourniquet.

18. A computer program product comprising stored instructions which when executed in a processor having outputs adapted to provide control signals to inflate and deflate a tourniquet, cause said processor to perform a method having the steps of:
(a) performing an inflation phase in which a tourniquet applied to a body part is inflated to a working pressure which exceeds the limb occlusion pressure (LOP) of said body part by an offset amount;
(b) measuring the time-varying pressure induced in said tourniquet by the vascular system of said body part during said inflation;
(c) deriving from said time-varying pressure a set of values for a characteristic of the induced pressure pulse in said tourniquet at a plurality of tourniquet pressures during the inflation phase;
(d) determining from said set of values a first threshold value intermediate between the value of the characteristic at the LOP and the value of the characteristic at the working pressure;
(e) monitoring the value of said characteristic during a working phase of the tourniquet while maintaining said working pressure; and
(f) upon determining that the value of said characteristic has reached or passed said first threshold, increasing the working pressure of the tourniquet.
